(19) United States
(12) Patent Application Publication
(10) Pub. No.: US 2005/0096482 A1
(43) Pub. Date: May 5, 2005

(54) Method of depolymerizing polyethylene terephthalate and process for producing polyester resin

(76) Inventors: Ryozo Tamada, Hyogo (JP); Yasuhiro Iguchi, Hyogo (JP); Nobu Yoshimura, Kanagawa (JP); Shinichi Otuka, Kanagawa (JP)

Correspondence Address: Fildes & Outland, Suite 2, 20916 Mack Avenue, Grosse Pointe Woods, MI, 48236, US

(21) Appl. No.: 10/502,681

(22) PCT Filed: Oct. 28, 2002
(86) PCT No.: PCT/JP02/11183

(30) Foreign Application Priority Data
Feb. 1, 2002 (JP) 200224881
Jun. 28, 2002 (JP) 2002188815

(51) Int. Cl. C07C 67/02

(52) U.S. Cl. 560/89

(57) ABSTRACT

A method of depolymerizing polyethylene terephthalate, and a method of manufacturing a polyester resin. When heating, melting and depolymerizing polyethylene terephthalate to be recycled, the heating, melting and depolymerization reaction of the polyethylene terephthalate to be recycled are carried out all at once using one or a plurality of extruders or using an extruder and a reactor provided at an outlet of the extruder. When manufacturing a polyester resin, the reactants are irradiated with microwaves, thus promoting the heating of the reactants, and promoting the esterification reaction.

### TECHNICAL FIELD

The present invention relates to a method of depolymerizing polyethylene terephthalate to be recycled, and a method of manufacturing a polyester resin. Hereinafter, polyethylene terephthalate shall be abbreviated to ‘PET’, and polyethylene terephthalate to be recycled shall be abbreviated to ‘R-PET’.

### BACKGROUND ART

Methods of obtaining an unsaturated polyester resin using R-PET as a raw material are publicly known.

As such a method, in general a method is adopted in which a glycol is charged into a reaction vessel, the R-PET is charged in divided amounts at a temperature below the boiling point of the glycol, thus carrying out glycolysis to produce anoligomer, and then a desired amount of an α,β-unsaturated polybasic acid (or acid anhydride thereof) is added and polycondensation is carried out to produce an unsaturated alkyd, and this unsaturated alkyd is dissolved in a styrene monomer which acts as a crosslinking agent.

Drawbacks of this method are that the temperature cannot be raised above the boiling point of the glycol and hence a long time is required for the depolymerization, and the R-PET can only be charged in in an amount such that the glycol:R-PET mass ratio is approximately 1:1, with it not being possible to increase the proportion of the R-PET added. The shorter the reaction time, the more advantageous in terms of cost, and moreover it is self-evident from the viewpoint of the use of waste and recycling that the higher the proportion of R-PET used, the better.

As a method solving the problem of increasing the proportion of R-PET added, a method has been proposed in which the R-PET is melted and a required amount of a tin-based catalyst or a titanium-based catalyst is added, and then a glycol is instilled therein, thus promoting the decomposition of the R-PET (JP-A-2000-7770, JP-A-11-60707, JP-A-2002-60474). Moreover, to shorten the melting time of the R-PET in the reaction vessel, it has been proposed to melt the R-PET at a temperature above the melting point thereof using a kneader, and then charge the R-PET into the reaction vessel (JP-A-2002-114839).

However, with the above methods, to maintain the molten state of the R-PET in the reaction vessel, the reaction vessel must be kept at above the melting point of the R-PET, and moreover the depolymerization reaction requires several hours.

With such a publicly known method, for example, R-PET flakes are charged into the reaction vessel, heating and melting are carried out, and to produce an oligomer having an average molecular weight of not more than 3000, at least 120 minutes at above the melting point of the R-PET is required for the heating and melting reaction, and furthermore at least 300 minutes is required for the depolymerization reaction.

### DISCLOSURE OF THE INVENTION

It is an object of the present invention to enable the depolymerization of R-PET to be carried out in a short time.

To attain the above object, the present inventors discovered that by charging R-PET into an extruder, and applying a shearing force at a low speed while heating the cylinder, and thus heating and melting the charged material and simultaneously uniformly kneading the charged material, a PET oligomer can be obtained, and this heating, melting and depolymerization reaction leads to the decomposition and depolymerization of the R-PET molecular chain originally aimed for, thus arriving at the present invention.

In the present invention, when heating, melting and depolymerizing R-PET, the heating, melting and depolymerization reaction of the R-PET are carried out all at once using one or a plurality of extruders or using an extruder and a reactor provided at an outlet of the extruder.

As a result, a great improvement in productivity and simplification of the process can be realized for the process of manufacturing an oligomer having an average molecular weight of not more than 3000 from R-PET, for example waste PET bottle flakes. The oligomer obtained can be used in the manufacture of resins such as unsaturated polyester resins synthesized based on the PET molecular framework.

According to the present invention, for heating and melting the R-PET, one or a plurality of extruders having a desired discharge amount are used, or an extruder and a reactor provided at an outlet of the extruder are used, the cylinder heating conditions are, for example, made to be in a range of 160 to 320° C. (preferably 220 to 280° C.), and the R-PET is subjected to a depolymerization reaction inside the extruder(s) and/or reactor, thus obtaining a PET oligomer. The PET oligomer is charged as a raw material into another reaction vessel, and additional glycol is added as required; to obtain an oligomer having an average molecular weight of not more than 3000 from the R-PET, the reaction in the extruder takes 10 to 20 minutes, and the further depolymerization reaction in the another reaction vessel takes 60 minutes, and hence a great reduction in time can be realized compared with publicly known art. Furthermore, by promoting the depolymerization reaction in the extruder, or additionally providing a reactor at the outlet of the extruder, the depolymerization reaction in a separate reaction vessel can be omitted. In the case of additionally providing a reactor at the outlet of the extruder, the overall process can be made yet shorter, with a time of only 30 to 40 minutes being required to obtain an oligomer having an average molecular weight of not more than 3000 from the R-PET. Moreover, according to the present invention, the melting temperature of the PET oligomer in the another reaction vessel can be made almost 100° C. lower than with a publicly known method, and moreover a high R-PET content can be maintained. Moreover, because the glycol is present, the efficiency of heat transfer into the raw material is also good.

According to the present invention, it is preferable to add a tin-based catalyst such as dibutyltin oxide or a titanium-based catalyst such as tetraisopropoxytitanate that facilitates the depolymerization to the raw material supplied into the extruder.

A melting reaction in an extruder and extrusion are used with urethane resins and so on in publicly known art, but in that case depolymerization is not an objective, but rather the art relates to a regeneration treatment method, and decomposition to produce a raw material is not carried out as in the present invention (JP-A-8-300352, JP2000-281831, etc.).

### BRIEF DESCRIPTION OF THE DRAWINGS

FIG. 1 shows schematically a constitution of a test apparatus for carrying out a method of depolymerizing polyethylene terephthalate according to the present invention; and

FIG. 2 shows schematically a constitution of a test apparatus for carrying out a method of manufacturing a polyester resin according to the present invention.

### BEST MODE FOR CARRYING OUT THE INVENTION

R-PET that can be used in the present invention is predominantly a material recycled from PET bottles, and in general has the form of flakes or pellets.

When the R-PET passes through an extruder while being heated, water contained in the R-PET does not have an adverse effect on the decomposition of the R-PET, but rather the presence of water has virtually no influence on the depolymerization of the R-PET. It is thus not necessary to dry the R-PET.

An extruder used in the present invention may be an ordinary single-screw or twin-screw extruder or any other extruder, so long as the raw materials can be heated and kneaded uniformly, and a shearing force can be applied thereto.

With the heating, melting and depolymerization reaction in the extruder, a tin-based catalyst such as dibutyltin oxide or a titanium-based catalyst such as tetraisopropoxytitanate can be added to the molten R-PET in the cylinder of the extruder, this being with an aim of facilitating the depolymerization in the R-PET depolymerization process, and furthermore a glycol of a type used as a component of the final product can be added. The catalyst and the glycol may be added by being supplied into the extruder separately, or predetermined amounts of the catalyst and the glycol may be added to the R-PET in advance, with the resulting mixture being charged into the extruder. In the former case, predetermined amounts of the catalyst and the glycol are supplied in relative to the extruded amount per unit time.

To promote the depolymerization reaction, it is also preferable to use a method in which a predetermined amount of the glycol is added, or predetermined amounts of the glycol and the catalyst are added, to the PET oligomer that has passed through the extruder, and the material is passed through the extruder a plurality of times, or a method in which the material is passed through a plurality of extruders linked together.

The reaction is controlled by controlling the heat in the cylinder of the extruder, whereby the state of the PET oligomer extruded is controlled. The extruded PET oligomer may be charged into another reaction vessel in a molten state, or may be solidified at ordinary temperature and then stored as a raw material.

In the case of providing a reactor at an outlet of the extruder, a reactor of any chosen constitution can be used, for example a tubular reactor, so long as the reactor can carry out heating, and the extruded PET oligomer can flow through the reactor in a molten and stirred state. Note that if a static mixer or the like is used, and homogeneous heating and decomposition are carried out, then a PET oligomer of better quality can be obtained.

Examples of catalysts that can be used in the present invention are: tin-based catalysts such as dibutyltin oxide, tin octylate and dibutyltin dilaurate; titanium alkoxides such astetrabutoxytitanate (TBT), tetraisopropoxytitanate (TPT) and tetraethoxytitanate; and zinc organic acid salts such as zinc acetate.

The amount used of the catalyst is 0.01 to 3 parts by mass, more preferably 0.1 to 1 part by mass, per 100 parts by mass of the R-PET. By adding such a decomposing catalyst in the heating, melting and depolymerization reaction in the extruder, glycolysis of the R-PET is markedly promoted.

The following types of glycol can, for example, be used in the present invention. That is, examples include ethylene glycol, diethylene glycol, propylene glycol, dipropylene glycol, neopentyl glycol, 2-methylpropanediol, 1,3-butanediol, 1,4-butanediol, 3-methylpentanediol, 1,2-butanediol, hydrogenated bisphenol A, bisphenol A ethylene oxide adjunct, and bisphenol A propylene oxide adjunct. Moreover, polyhydric alcohol having three or more functional groups such as polyethylene glycol, polypropylene glycol, glycerol, trimethylethane, trimethylpropane or pentaerythritol can be used. A combination thereof can also be used.

The amount used of the glycol is made to be 0.1 to 10 mol per 1 mol of the condensed unit of the R-PET (taking the repeat unit to be 1 mol). A desired amount of the glycol may be added in the extruder, and the product may be subjected to a further depolymerization reaction in another reaction vessel. Alternatively, the required amount maybe added in the extruder, with the depolymerization reaction in another reaction vessel being omitted.

When manufacturing a resin such as an unsaturated polyester resin according to the present invention, the R-PET is heated, melted and depolymerized using one or a plurality of extruders, or using an extruder and a reactor provided at an outlet of the extruder, thus obtaining an oligomer of average molecular weight not more than 3000. In the case of manufacturing an unsaturated polyester resin, taking the product PET oligomer as a raw material, or after carrying out a further depolymerization reaction by adding a desired amount of the glycol to this oligomer in another reaction vessel, an α,β-unsaturated polybasic acid (or acid anhydride thereof) is added, and as necessary another saturated or unsaturated polybasic acid (or acid anhydride thereof) is also used, and polycondensation is carried out.

In the case of manufacturing an unsaturated polyester resin, in general maleic anhydride or fumaric acid can be used as the α,β-unsaturated polybasic acid (or acid anhydride thereof). The use of another saturated or unsaturated polybasic acid (or acid anhydride thereof) is at one's liberty. After one or a plurality of such α,β-unsaturated acids or acid anhydrides thereof has/have been added and polycondensation has been completed, dissolution is carried out in a desired monomer, whereby the unsaturated polyester resin is obtained.

This monomer is generally styrene. Other than this, methyl methacrylate, diallyl phthalate or the like can also be used in accordance with the application.

The proportions used of the R-PET, the glycol and the unsaturated acid will vary according to the application, but are approximately 10 to 80 mol % of the R-PET, 20 to 90 mol % of the glycol, and 10 to 80 mol % of the unsaturated acid.

The number of mols of the PET is calculated taking the structure of the following formula as 1 mol.

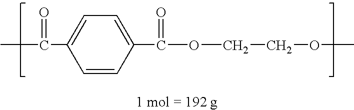

An unsaturated polyester resin obtained as described above is useful in various applications. Any of various additives, for example thermoplastic polymers/oligomers, colorants, mold release agents, stabilizers and so on, can of course be used in accordance with the application.

Resins other than an unsaturated polyester resin as described above can also be manufactured in accordance with the present invention.

In general, when manufacturing an unsaturated polyester resin, a saturated dibasic acid such as phthalic anhydride, an unsaturated polybasic acid such as maleic anhydride or fumaric acid, and a glycol such as ethylene glycol or propylene glycol are subjected to polycondensation to produce an unsaturated alkyd, and then the unsaturated alkyd is dissolved in a polymerizable vinyl monomer such as styrene.

With publicly known art, a long time is also required to manufacture such an unsaturated polyester resin. According to the present invention, a polyethylene terephthalate resin that has been depolymerized as described earlier can be used instead of a dimethyl terephthalate component, and this polyethylene terephthalate resin can be used as a partial replacement for the resin component. However, in this case, the reaction time is again long as above, and moreover the dissolution of the waste PET bottle flakes takes time, and hence an even longer time is required.

To cope with this, in the present invention, when synthesizing a polyester resin through an esterification reaction using a saturated dibasic acid, an unsaturated polybasic acid and a glycol, microwaves are irradiated onto the reactants, thus raising the temperature of the reactants, and promoting the esterification reaction.

That is, in the present invention, a polyester resin can be produced efficiently by ‘irradiating the reactants with microwaves to promote heating of the reactants’ and ‘irradiating the reactants with microwaves to promote the esterification reaction’.

Consequently, according to the present invention, the reaction can be completed in a short time, for example overall the reaction can be completed in approximately one third to one quarter of the time compared with a publicly known method.

Furthermore, in the present invention, when depolymerizing R-PET using a glycol or the like, and charging an unsaturated polybasic acid such as maleic anhydride into the depolymerization product and heating to carry out an esterification reaction, the temperature of the depolymerization product and the unsaturated polybasic acid may be raised by irradiating with microwaves.

Furthermore, in the present invention, when depolymerizing R-PET using a glycol or the like to obtain a depolymerization product, the molecular weight of the depolymerization product may be reduced by irradiating the depolymerization product with microwaves.

Furthermore, in the present invention, when using a glycol or the like on R-PET to depolymerize this R-PET, and charging an unsaturated polybasic acid such as maleic anhydride into the depolymerization product and heating to carry out an esterification reaction, the esterification reaction may be promoted by irradiating the depolymerization product with microwaves.

That is, when manufacturing a tere-type unsaturated polyester resin, according to the present invention, R-PET such as waste PET bottle flakes or waste PET bottle pellets can be used instead of dimethyl terephthalate. In this case, specifically, the unsaturated polyester resin can be manufactured by adding a glycol to the R-PET such as waste PET bottle flakes, producing a PET oligomer depolymerized down to a degree of polymerization of, for example, 800 or less, and adding a predetermined amount of maleic anhydride to the PET oligomer and carrying out an esterification reaction. Here, when the R-PET is dissolved in the glycol and the depolymerization reaction is carried out, with publicly known art a reaction time of 4 to 6 hours is required.

In the present invention, in this series of reactions, by ‘irradiating the PET oligomer that is the depolymerization product with microwaves to raise the temperature of the irradiated material and promote the depolymerization reaction’, and further ‘when charging the unsaturated polybasic acid such as maleic anhydride into the depolymerization product and carrying out an esterification reaction, irradiating the reactants with microwaves to promote the esterification reaction’, the overall time required when manufacturing the polyester resin from the R-PET can be greatly shortened. As a result, the polyester resin can be manufactured very efficiently.

Microwaves are widely used in general in household microwave ovens. In the present invention, microwaves having a frequency of 2450 MHz used in a household microwave oven are used in a direct heating method or a synthesis reaction promoting method when manufacturing an unsaturated polyester resin, and can also be used in a method of promoting a depolymerization reaction carried out by adding a glycol to waste PET such as waste PET bottle flakes, and a method of promoting the reaction when adding an unsaturated polybasic acid such as maleic anhydride to the depolymerization product oligomer and carrying out an esterification reaction.

As the method of heating and reacting in each of the above stages, hitherto, a method in which the reactants are not heated themselves but rather the vessel containing the reactants is heated electrically, a method in which heating is carried out by circulating a heating medium such as heating oil, or the like has been used in general.

In contrast with this, the present inventors discovered that microwaves can be used in promoting reaction by being irradiated directly onto esterification reactants, and can be used in directly heating the reaction system by directly irradiating the reactants with microwaves in a reaction of using a glycol to depolymerize waste PET bottle flakes obtained through pulverizing waste PET bottles and an esterification reaction for resynthesizing an unsaturated polyester resin using the depolymerization product as a raw material, and that in addition to raising the temperature of the reactants in the esterification reaction or the depolymerization reaction, there is also a very large effect on the reaction itself, and hence microwaves can be used in promoting the esterification reaction or the depolymerization reaction, thus accomplishing the present invention.

In the present invention, as the type of microwaves, 2450 MHz is generally used as described above, but there are no particular limitation on the frequency. A 915 MHz generator is used in thawing of food, and this can also be used in the present invention.

As the esterification reaction components, phthalic anhydride, isophthalic acid, terephthalic acid, tetrahydrophthalic anhydride, methyltetrahydrophthalic anhydride, endomethylenetetrahydrophthalic anhydride, adipic acid, HET acid, tetrabromophthalic anhydride and so on can be used as the saturated dibasic acid, and maleic anhydride, fumaric acid, itaconic acid and so on can be used as the unsaturated polybasic acid.

Moreover, examples of the glycol are as earlier. For the glycol, a heat-receiving effect as a heated substance upon being irradiated with microwaves can be expected, and promotion of an esterification reaction or the like can be expected at the same time.

When manufacturing a polyester resin according to the present invention, irradiation with microwaves is carried out from the initial heating stage, and after the temperature has been raised to 160° C., generation of reaction water is observed upon a further increase in temperature. The irradiation with microwaves is thus continued while carrying out a water removal operation, and then the operation is continued while reducing the pressure, whereby the synthesis reaction from the time at which the reaction water starts to be generated up to the desired acid value being obtained can be completed in approximately 2.5 hours. Overall the reaction can thus be completed in approximately one third to one quarter of the time compared with a publicly known method. The effect of the irradiation with microwaves is not mere heating, but rather it is found that there is also an effect on the esterification reaction itself.

During the esterification reaction, by similarly irradiating with microwaves, the reaction can be promoted, and hence the reaction can be completed in a short time. As a result, the alkyd component produced is dissolved in styrene monomer or the like, whereby the final unsaturated polyester resin can be obtained.

Unsaturated polyester resins having terephthalic acid as a component thereof become cured resins having excellent water resistance, chemical resistance and toughness, and are useful in various FRP (fiber reinforced plastic) fields, being widely used in general. In particular, in recent years there have been advances in PET bottle recycling technology and the product quality has improved, and trials into glycolyzing recycled R-PET as described above to produce a raw material for manufacturing an unsaturated polyester resin have been carried out with vigor.

To make R-PET into a raw material for an unsaturated polyester resin, the high-molecular-weight R-PET must be glycolyzed by being ‘boiled’ with a glycol. However, after the unsaturated polyester resin has been manufactured by glycolyzing the R-PET, there is a tendency for the unsaturated polyester resin to become cloudy (albeit to different extents) over time. This phenomenon is not only observed with resins using R-PET, but rather also occurs with unsaturated polyester resins manufactured using terephthalic acid as a resin raw material.

The cause of the cloudiness has been found to be a small amount of free terephthalic acid. In the case that the glycolysis is insufficient, or in the case that the amount of the glycol used is low and hence is insufficient to decompose the R-PET sufficiently, an R-PET oligomer is produced, and this may also cause cloudiness. Even with a cloudy unsaturated polyester resin, hardly any effect is seen on the principal material properties of the cured resin, but the appearance is markedly degraded, and hence the commercial value of the resin is degraded.

The present inventors carried out various studies into solving the clouding problem of unsaturated polyester resins having terephthalic acid as a component thereof, and as a result discovered that using an alkali metal organic acid salt is surprisingly very effective, and that so long as no error is made in the amount added or the timing of the addition, the clouding over time of an unsaturated polyester resin using PET or an unsaturated polyester resin using terephthalic acid can be completely prevented, thus accomplishing the present invention.

That is, in the present invention, an alkali metal organic acid salt is added to an unsaturated polyester resin containing terephthalic acid, thus preventing clouding of the polyester resin.

According to the present invention, the alkali metal organic acid salt is preferably a sodium organic acid salt or a potassium organic acid salt.

Moreover, according to the present invention, the unsaturated polyester resin containing terephthalic acid can be made to be one synthesized using R-PET as a raw material.

There are no particular limitations on alkali metals that can be used in the present invention, but the effect tends to be somewhat inferior with lithium, and rubidium and cesium are expensive and thus are not generally used. Sodium and potassium are thus practical, with potassium being better, and hence a potassium organic acid salt is most suitable for the objective of the present invention.

Regarding organic acid salts of other metals, for example in the case of using an alkaline earth metal, an organic acid salt of calcium, magnesium, strontium, barium or the like, a slight effect is observed with some, but this falls far short of what is required for practical use. Organic acid salts of other heavy metals have no effect.

There are no particular limitations on the type of the organic acid, but due to the necessity of dissolving the unsaturated polyester resin component in styrene, for example naphthenic acid, octylic acid (2-ethylhexanoic acid) and so on are preferable.

The amount used of the alkali metal organic acid salt varies according to such conditions as follows: assuming that R-PET is used, to what proportions the R-PET is mixed with the glycol; whether another polybasic acid (or acid anhydride thereof) is used with the terephthalic acid; or whether polycondensation is carried out with a glycol as well as an α,β-unsaturated polybasic acid, without using another polybasic acid. In general, for example in the case of using potassium octylate, the proportion used is 0.01 to 5 parts by mass, more preferably 0.1 to 0.5 parts by mass, per 100 parts by mass of the unsaturated polyester resin. At less than 0.01 parts by mass, the addition no longer has any observable effect. If more than 5 parts by mass is added, the additional amount will no longer have any observable effect, and moreover the tendency for the material properties of the resin to be degraded will become marked.

The timing of the addition is preferably after the unsaturated polyester has been synthesized and the unsaturated polyester resin has been produced by dissolving in styrene, with immediately after the manufacture of the resin being best.

The alkali metal organic acid salt does not have adverse effects on the material properties of the cured resin if the proportion added is as above; the alkali metal organic acid salt may exhibit an action of promoting the curing of the liquid resin.

### EXAMPLES

### Example 1

Extruder: ‘S-1’ counter-rotating twin-screw extruder made by Kurimoto Iron Works Co., Ltd.

Extruder temperature: 220 to 280° C.

Materials: (i) Waste PET bottle flakes (R-PET): made by Yono PET Bottle Recycle Co., Ltd.

(ii) Propylene glycol
(iii) Catalyst: Dibutyltin oxide
   Method

(1) The extruder was set to a predetermined temperature, and a material that had been obtained by adding the propylene glycol and the dibutyltin oxide to the R-PET in advance was supplied in.

(2) The molten R-PET that had been subjected to a depolymerization reaction upon passing through the extruder was solidified at room temperature. For each addition, the molecular weight and the melting point of the solidified material were measured, whereupon both dropped for each addition as in Table 1 below.

TABLE 1

| | | Molecular | Melting |
|---|---|---|---|
| | Details of addition | weight | point (° C.) |
| a) | R-PET only | 10900 | 240-250 |
| b) | 25 mass % of propylene glycol added to | 6350 | 230-240 |
| | R-PET | | |
| c) | 25 mass % of propylene glycol and | 2450 | 220-230 |
| | 0.3 mass % of dibutyltin oxide | | |
| | added to R-PET | | |
| d) | c) pulverized, 25 mass % of propylene | 1880 | 170-180 |
| | glycol added, and then charged into | | |
| | extruder again | | |
| e) | d) pulverized, 25 mass % of propylene | 1680 | 140-150 |
| | glycol added, and then charged into | | |
| | extruder again | | |

(3) Next, an unsaturated polyester resin was synthesized using the PET oligomer obtained, namely PET oligomer e) in Table 1, as a raw material. Specifically, 200 g of PET oligomer e) and 30 g of propylene glycol were charged into a 1 L separable flask equipped with a stirrer, a reflux condenser, a dropping funnel and a gas introducing pipe-possessing thermometer, and glycolysis was carried out for 1 hour at 210 to 220° C. under a stream of nitrogen. As a result, the average molecular weight dropped to approximately 600 to 800. The condenser was then changed over to a fractional distillation type, the temperature was reduced to 210° C., 130 g of maleic anhydride was added, and esterification was carried out for 2 hours, and then a polycondensation reaction was carried out for 1 hour under a reduced pressure of approximately 30 Torr. 0.17 g of hydroquinone was then added once the acid value had reached 31, 338 g of styrene was added under a stream of air at a temperature of 140° C., and dissolution was carried out to uniformity. As a result, an unsaturated polyester resin was obtained as a pale yellowish brown liquid.

4) Moreover, using a similar apparatus, 200 g of PET oligomer e), 30 g of propylene glycol and 130 g of maleic anhydride were charged in, and without carrying out the additional glycolysis, esterification was carried out for 2 hours at 210° C. under a stream of nitrogen, and then a polycondensation reaction was carried out for 1 hour under a reduced pressure of approximately 30 Torr. 0.17 g of hydroquinone was then added, 338 g of styrene was added under a stream of air at a temperature of 140° C., and dissolution was carried out to uniformity. As a result, an unsaturated polyester resin was obtained as a somewhat cloudy pale yellowish brown liquid.

### Example 2

Extruder: ‘SRV-P40/30’ single-screw extruder made by Nihon Yuki Co., Ltd., L/D=22

Extruder temperature: 220 to 280° C.

Materials: (i) Waste PET bottle flakes (R-PET): made by Yono PET Bottle Recycle Co., Ltd.

(ii) Propylene glycol
(iii) Catalyst: Dibutyltin oxide
   Method

(1) The extruder was set to a predetermined temperature, and raw materials as above were supplied in.

(2) The molten R-PET that had passed through the extruder was solidified at ordinary temperature. For each addition, the molecular weight and the melting point of the solidified material were measured, whereupon both dropped for each addition as in Table 2 below.

TABLE 2

| | | Molecular | Melting |
|---|---|---|---|
| | Details of addition | weight | point (° C.) |
| a) | R-PET only | 15400 | 240-250 |
| b) | 0.3 mass % of dibutyltin oxide and | 3050 | 220-230 |
| | 5 mass % of propylene glycol added | | |
| | to R-PET in advance | | |
| c) | 0.3 mass % of dibutyltin oxide added to | 6480 | 230-240 |
| | R-PET in advance, and 5 mass % | | |
| | of propylene glycol added from | | |
| | part way along extruder | | |

### Example 3

Extruder: ‘PMT 47-III’ double-screw extruder made by IKG, L/D=30

Extruder temperature: 220 to 280° C.

Materials: (i) Waste PET bottle flakes (R-PET): made by Yono PET Bottle Recycle Co., Ltd.

(ii) Propylene glycol
(iii) Catalyst: Dibutyltin oxide or tetraisopropoxytitanate
   Method

(1) The extruder was set to a predetermined temperature, and raw materials as above were supplied in.

(2) Regarding the details of the addition, 0.3 mass % of the catalyst was added to the R-PET in advance, and 50 mass % of propylene glycol was supplied in from part way along the extruder using a metering pump.

(3) The molten R-PET that had passed through the extruder was a white solid at ordinary temperature. The molecular weight and the melting point of the solidified material were measured to be as in Table 3 below.

TABLE 3

| | | Molecular | Melting |
|---|---|---|---|
| | Details of addition | weight | point (° C.) |
| a) | 0.3 mass % of dibutyltin oxide added to | 920 | 100-140 |
| | R-PET in advance, and 50 mass % of | | |
| | propylene glycol added from part way | | |
| | along extruder | | |
| b) | 0.3 mass % of tetraisopropoxytitanate | 1060 | 120-160 |
| | added to R-PET in advance, and 50 mass | | |
| | % of propylene glycol added from part | | |
| | way along extruder | | |

(4) The PET oligomer obtained, namely PET oligomer b) in Table 3, propylene glycol and maleic anhydride were charged in simultaneously, esterification was carried out for 2 hours at 210° C. under a stream of nitrogen, and then a polycondensation reaction was carried out for 1 hour under a reduced pressure of approximately 30 Torr. Hydroquinone was then added, styrene was added under a stream of air at a temperature of 140° C., and dissolution was carried out to uniformity; as a result, an unsaturated polyester resin was obtained as a slightly cloudy pale yellowish brown liquid.

### Example 4

Extruder: ‘KZW 15’ co-rotating twin-screw extruder made by Technovel Corporation, L/D=75

Reactor: Static mixer made by Noritake Company

Temperature: 220 to 280° C.

Materials: (i) Waste PET bottle flakes (R-PET): made by Chukyo Niyaku PET Bottle Recycling Plant Co., Ltd.

(ii) Propylene glycol
(iii) Catalyst: Dibutyltin oxide
   Method

(1) The extruder was set to a predetermined temperature, and 0.3 mass % of the catalyst was added to the R-PET in advance, and 50 mass % of propylene glycol was supplied in from part way along the extruder cylinder using a metering pump.

(2) The static mixer, which was heated to a predetermined temperature, was installed on the outlet of the extruder, and the molten R-PET that had passed through the extruder was passed through the static mixer.

(3) As a result, a PET oligomer was obtained in the form of a slurry. The average molecular weight in this case was 600 to 800.

(4) Next, an unsaturated polyester resin was synthesized using the PET oligomer obtained as a raw material. Specifically, the PET oligomer and maleic anhydride were charged in simultaneously, esterification was carried out for 2 hours at 210° C. under a stream of nitrogen, and then a polycondensation reaction was carried out for 1 hour under a reduced pressure of approximately 30 Torr. Hydroquinone was then added, styrene was added under a stream of air at a temperature of 140° C., and dissolution was carried out to uniformity; as a result, an unsaturated polyester resin was obtained as a pale yellowish brown liquid.

### Example 5

Extruder: ‘KZW 15’ co-rotating twin-screw extruder made by Technovel Corporation, L/D=75

Tubular reactor: Steel pipe (filled with rushing rings in one section)

Temperature: 220 to 280° C.

Materials: (i) Waste PET bottle flakes (R-PET): made by Chukyo Niyaku PET Bottle Recycling Plant Co., Ltd.

(ii) Propylene glycol
(iii) Catalyst: Dibutyltin oxide

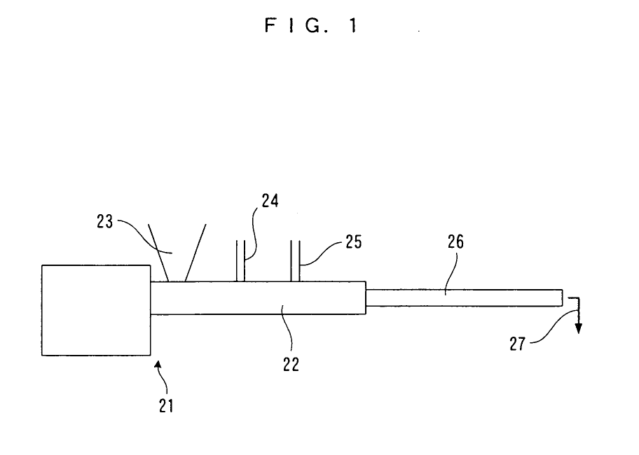

FIG. 1 shows schematically the constitution of the test apparatus. 21 denotes the extruder, which has a cylinder 22. The cylinder 22 is provided with a waste PET bottle flake supply port 23, a catalyst supply port 24, and a propylene glycol supply port 25. The steel pipe 26 is connected as a reactor tube to an outlet of the cylinder 22 of the extruder 21.

Method

(1) The extruder 21 was set to a predetermined temperature, and without using the catalyst supply port 24, 0.3 mass % of the catalyst was added to the R-PET in advance, and the resulting mixture was supplied into the cylinder 22 from the supply port 23. Moreover, 50 mass % of propylene glycol was supplied in using a metering pump from the supply port 25 part way along the cylinder 22 of the extruder 21.

(2) The steel pipe 26 (tubular reactor) installed on the outlet of the cylinder 22 of the extruder 21 was heated to a predetermined temperature, and the R-PET that had passed through the extruder 21 was passed through the steel pipe 26.

(3) As a result, a PET oligomer 27 was obtained in the form of a slurry. The average molecular weight in this case was 600 to 800.

As described above, using one or a plurality of extruders, or using an extruder and a reactor provided at an outlet of the extruder, the heating, melting and depolymerization reaction of the R-PET were carried out all at once, whereby a PET oligomer having an average molecular weight of not more than 3000 could be obtained efficiently. Moreover, by using this PET oligomer as a raw material, a great improvement in productivity and simplification of the process could be realized for the overall process of manufacturing an unsaturated polyester resin or another resin from waste PET bottle flakes.

### Example 6

(Preliminary Heating Test Using Household Microwave Oven)

Device used: ‘NE-8500’ made by Matsushita Electric Industrial Co, Ltd.

Rated voltage: 100 V
Rated power consumption: 990 W
Rated microwave output: 500 W
Microwave frequency: 2450 MHz
Chamber dimensions: Width 300 mm×depth 305 mm×height 195 mm
   Test Method

48.7% of propylene glycol was added per 100% of waste PET bottle flakes in terms of mass, 0.3% of dibutyltin oxide was further added as a catalyst, and depolymerization was carried out at 260 to 290° C. using an extruder, thus obtaining a waste PET bottle flake oligomer. 350 g and 450 g of this waste PET PET bottle flake oligomer were then each put into a 500 mL glass beaker, and irradiation with microwaves was carried out using the above device. As a result, the heating data shown in Table 4 below were obtained. The heating efficiency of the microwaves using this method was as follows, and hence it was found that this method is effective as a direct heating method with the above reactant system. That is, the heating efficiency exhibited a value of over 80% initially, and approximately 40% even after the rise in temperature, and hence the heating efficiency was found to be sufficient. Note that the heating efficiency is defined through the following formula.
Heating efficiency=amount of heat energy received by heated material/amount of heating energy of microwaves

TABLE 4

| | With 350 g of | | With 450 g of | |
|---|---|---|---|---|
| | heated material | | heated material | |
| | Temperature | | Temperature | |
| Total heating | of heated | Heating | of heated | Heating |
| time (s) | material (° C.) | efficiency | material (° C.) | efficiency |
| 0 | 85 | — | 97 | — |
| 30 | 115 | 1.25 | 113 | 1.00 |
| 60 | 135 | 0.83 | 127 | 0.88 |
| 90 | 148 | 0.54 | 140 | 0.81 |
| 120 | — | — | 146 | 0.38 |
| 150 | 171 | 0.48 | 153 | 0.44 |
| 210 | 189 | 0.38 | 166 | 0.41 |
| 270 | 200 | 0.23 | 180 | 0.44 |
| 300 | — | — | 184 | 0.13 |
| 360 | — | — | 200 | 0.50 |

### Example 7

(Depolymerization by Irradiation with Microwaves)

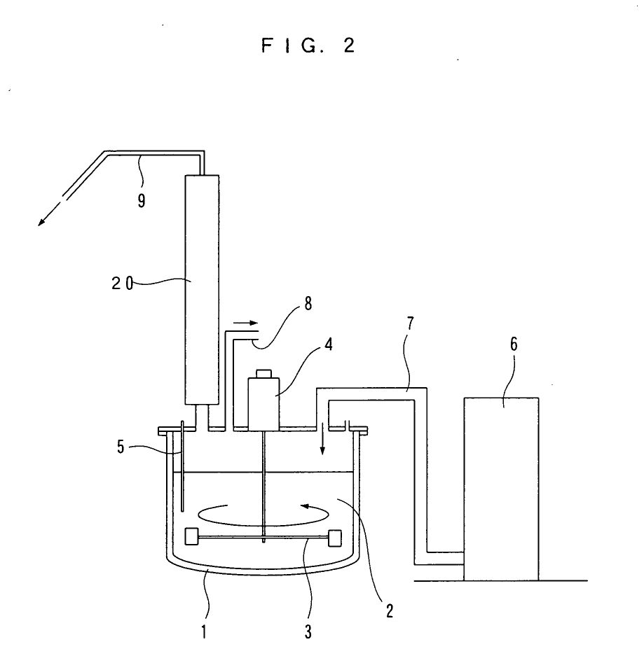

FIG. 2 shows schematically the constitution of the test apparatus. Here, 1 denotes an experimental reaction pot, which can house reactants 2 therein, and is provided with a rotational stirrer 3 for stirring the reactants 2. 4 denotes a rotational driving source for the stirrer 3. 5 denotes a thermometer for measuring the temperature of the reactants 2. 6 denotes a microwave generator, which is able to irradiate microwaves onto the reactants 2 inside the reaction pot 1 via a waveguide 7. Specifically, the reactants 2 inside the reaction pot 1 are not heated indirectly by heating the reaction pot 1, but rather microwaves can be irradiated directly onto the reactants 2. 8 denotes a vacuum suction passage, using which the pressure inside the reaction pot 1 can be reduced. 9 denotes a pipeline for removing water, which is provided with a condenser 20, whereby water inside the reaction pot 1 can be discharged to the outside.

48.7% of propylene glycol, and 0.3% of dibutyltin oxide as a depolymerization catalyst, per 100% of waste PET bottle flakes in terms of mass were charged into an extruder, and the waste PET bottle flakes were depolymerized to a molecular weight of approximately 1500, thus producing an oligomer.

4.4 kg of the oligomer was then charged into a 10 L experimental reaction pot 1 as shown in FIG. 2, and while stirring with the stirrer 3, microwaves of frequency 2450 MHz from the microwave generator 6 were irradiated from above the liquid surface in the reaction pot 1, thus carrying out an experiment into promoting the depolymerization. As a result, with a microwave irradiation time of 30 minutes from approximately 180° C., as shown in Table 5 below, it was possible to reduce the molecular weight to approximately half that before the reaction.

TABLE 5

| Microwave | Temperature | Average |
|---|---|---|
| irradiation | of contents | molecular |
| time (min) | (° C.) | weight (mw) |
| 0 | 178 | 1403 |
| 5 | 171 | |
| 10 | 179 | |
| 15 | 189 | |
| 18 | 195 | |
| 20 | 194 | |
| 25 | 195 | |
| 30 | 203 | 728 |

Molecular weight before irradiating with microwaves: 1482
Molecular weight measurement method: GPC analysis
Equipment used:
Microwave generator: ‘TMG-491C’ made by Yamamoto Vinita Co., Ltd. 5 kW output
10 L experimental pot: Internal volume 11536 cm³
Cavity watt density=5000/11536=0.43 W/cm³

### Comparative Example 1

(Depolymerization Using Publicly Known Heating Method)

4.1 kg of waste PET bottle flakes and 12.2 g of dibutyltin oxide as a reaction catalyst were charged into an extruder the cylinder of which had been heated to 290 to 300° C., and melting and kneading were carried out. The mixture was then charged into a reaction tank that had been heated to 250° C., and was stirred thoroughly.

After that, propylene glycol was charged in carefully a little at a time such that the temperature inside the reaction tank did not drop below 200° C. This operation required approximately 40 minutes. After that, the system was held for 60 minutes with the reaction tank maintained at 200° C., and then the contents were sampled, and the molecular weight was measured, whereupon an average molecular weight of 800 was obtained. The reaction was then continued for a further 1 hour while holding at 200° C. As a result, the molecular weight dropped to 690.

The time required for the depolymerization was thus 100 to 220 minutes, which is more than 3 times that in Example 7.

### Example 8

(Esterification by Irradiating with Microwaves)

4.4 kg of the oligomer of Example 7 (180° C.) obtained by depolymerizing waste PET bottle flakes with propylene glycol, and 1.7 kg of maleic anhydride were charged into a 10 L experimental reaction pot 1 as shown in FIG. 2, stirring was carried out for 15 minutes, and then 5 kW of microwaves of frequency 2450 MHz were irradiated from above the reaction liquid, thus increasing the temperature to 200° C. Once the temperature had reached 200° C., the water generated was discharged out of the system using the condenser 20, and moreover the pressure was reduced to 2660 Pa (20 Torr) using a vacuum pump, thus promoting the removal of water. After holding this state for 15 minutes, the acid value was measured, and was found to be 79. Holding the vacuum and temperature, irradiation with microwaves was further carried out for 15 minutes, and then the acid value was measured again, and was found to be 28. After irradiating with microwaves for a further 15 minutes, the acid value was measured again, and was found to be 13.6. At this point, the irradiation with microwaves was terminated, thus ending the reaction.

Next, 40% in terms of mass of a styrene monomer was charged in, and mixing and stirring were carried out to produce an unsaturated polyester resin. Here, an unsaturated polyester resin having a pale yellow appearance and a viscosity of 3.2 poise was obtained; this unsaturated polyester resin was the same as in the case of ordinary heating. Here, 45 minutes was taken from the charging in of the materials up to the completion of the alkyd reaction, and the rate of decrease of the acid value was faster than with an ordinary heating reaction, and hence it was clear that the esterification reaction proceeded more quickly.

| Equipment used: | | |
|---|---|---|
| Microwave generator: | ‘TMG-491C’ made by Yamamoto | |
| | Vinita Co., Ltd. | |
| | Output | 5 kW |
| | Frequency | 2450 MHz |
| 10 L experimental pot: | Internal volume 11536 cm³ | |
| | Cavity watt density = 5000/11536 = | |
| | 0.43 W/cm³ | |
| Resin properties | | |
| Hardening characteristics | Gelation | 64 min |
| | Hardening | 97 min |
| | Exotherm temperature | 129° C. |
| | Barcol hardness | 42 |

Resin concrete test pieces (40×40×160 mm rectangular blocks, resin content 14%) were manufactured from the unsaturated polyester resin obtained, and the performance was measured through bending tests. The results are shown in Table 6 below.

TABLE 6

| | Rupture stress | Flexural modulus | Rupture |
|---|---|---|---|
| | (kg/mm²) | (kg/mm²) | strain (%) |
| 1 | 3.41 | 2929.7 | 0.1230 |
| 2 | 3.49 | 2929.7 | 0.1159 |
| 3 | 3.18 | 2929.7 | 0.1088 |
| Mean | 3.36 | 2929.7 | 0.1159 |

### Comparative Example 2

(Esterification Using Publicly Known Heating Method)

A depolymerized oligomer (6090 g) was produced through reaction using the method of Comparative Example 1 in a 10 L experimental reaction pot 1 as shown in FIG. 2, and then 2310 g of maleic anhydride was charged in at 160° C. and heating was carried out. After stirring, the generation of water was observed after 10 minutes at 180° C., and hence removal of water using the condenser 20 was then commenced. In this state, the temperature was further raised to 200 to 203° C., and this state was maintained for 4 hours. A sample was then taken, and an acid value of 48 was obtained. A vacuum state of 3990 to 5320 Pa (30 to 40 Torr) was then created, and the reaction was continued for 1 hour 30 min, and then the acid value was measured again. At this time, an acid value of 28 was obtained. It was decided to end the reaction at this time, and hence the heating was stopped. 45 minutes later, 5600 g of styrene was then charged in at 140° C., and stirring was carried out, thus producing a pale yellow unsaturated polyester resin.

5 hours 30 min was required from the charging in of the materials up to the completion of the alkyd synthesis.

Resin properties

| Hardening characteristics | Gelation | 104 min |
|---|---|---|
| | Hardening | 159 min |
| | Exotherm temperature | 68° C. |
| | Barcol hardness | 39 |

### Example 9

(Esterification Reaction by Irradiating with Microwaves)

A mixture as follows was charged into a 10 L experimental reaction pot 1 as shown in FIG. 2, and trial synthesis of an unsaturated polyester resin was carried out under irradiation with microwaves. That is, 2400 g of phthalic anhydride, 2592 g of propylene glycol and 1590 g of maleic anhydride (total 6582 g) were charged into the reaction pot 1, and while passing in nitrogen gas, microwaves were irradiated directly onto the raw material mixture from the outset, and using an electric heater of the reaction pot 1 at the same time, heating was carried out. Here, 30 minutes was taken to reach 170° C. This temperature was held for 30 minutes, whereupon generation of water started, and hence removal of water using the condenser 20 was commenced.

After that, irradiation with microwaves was carried out for 30 minutes, thus raising the temperature to 200° C., and then the acid value was measured, and was found to be 128. Holding at 200° C. while irradiating with microwaves, the situation regarding the drop in the acid value (the reaction level) was then checked. As shown in Table 7 below, after a further 1 hour 45 min, an acid value of 56 was obtained. According to this, totaling the time of heating and maintaining the temperature from the start, it was possible to synthesize an alkyd having an acid value of 56 in 3 hours 15 min, and hence compared with the reaction system of Comparative Example 3 described below in which irradiation with microwaves was not carried out, it was possible to obtain a product of the same level of quality with approximately one quarter of the reaction time. Specifically, an unsaturated polyester resin having a pale yellow appearance and a viscosity of 2.6 poise was obtained. In the above, vacuum treatment was carried out after 2 hours 30 min had elapsed from the start.

Equipment used:

| | |
|---|---|
| Microwave generator: | ‘TMG-491C’ made by |
| | Yamamoto Vinita Co., Ltd. |

| | |
|---|---|
| Output | 5 kW |
| Frequency | 2450 MHz |

10 L experimental pot: Internal volume 11536 cm³

Cavity watt density=5000/11536=0.43 W/cm³

TABLE 7

| | Temper- | Micro- | | | Incident | |
|---|---|---|---|---|---|---|
| Time | ature | wave | | | current | Reflected |
| elapsed | inside pot | irradiation | Reaction | Acid | value | current |
| (hr) | (° C.) | time (hr) | water | value | (A) | value (A) |
| 0 | 38 | — | — | — | — | — |
| 0.50 | 175 | 0.5 | — | — | 0.64 | 0.26-0.3 |
| 1.00 | 163 | — | Started to | — | — | — |
| | | | be | | | |
| | | | generated | | | |
| 1.50 | 210 | 0.5 | Removed | 128 | 0.66 | 0.22 |
| 1.75 | 204 | — | Removed | — | — | — |
| 2.00 | 210 | 0.25 | Removed | 106 | 0.6 | 0.2 |
| 2.50 | 196 | — | Vacuum | — | — | — |
| | | | treatment | | | |
| 2.75 | 209 | 0.25 | Vacuum | 63 | 0.6 | 0.18 |
| | | | treatment | | | |
| 3.00 | 209 | — | Vacuum | — | — | — |
| | | | treatment | | | |
| 3.25 | 205 | 0.25 | Vacuum | 56 | 0.42 | 0.12 |
| | | | treatment | | | |

### Comparative Example 3

(Esterification Using Publicly Known Method)

A mixture as in Example 9 was charged into a 10 L experimental reaction pot 1 as shown in FIG. 2, and while passing in nitrogen gas, heating was carried out for 3 hours 15 min until the temperature reached 80 to 90° C. At this time, stirring became possible, and hence stirring was commenced. Heating was carried out for a further 2 hours 15 min, thus raising the temperature to 170° C. In this state, water started to be generated, and hence removal of water using the condenser 20 was commenced. After that, heating was carried out for 2 hours, thus raising the temperature to 200° C., and then the acid value was measured. The temperature was held at 200° C. for a further 1 hour 30 min, and then vacuum treatment was commenced. Regarding the relationship between the time held at 200° C. and the acid value, as shown in Table 8 below, the acid value reached 51 after a further 4 hours 30 min had elapsed, and hence totaling the time of heating and maintaining the temperature from the start, it was finally possible to synthesize an alkyd having an acid value of 51 in 12 hours.

Next, 40% of a styrene monomer was charged in, and mixing and stirring were carried out to produce an unsaturated polyester resin. As a result, an unsaturated polyester resin having a pale yellow appearance and a viscosity of 2.3 poise was obtained.

TABLE 8

| Time | Temperature | | |
|---|---|---|---|
| elapsed | inside pot | Reaction | |
| (hr) | (° C.) | water | Acid value |
| 0 | 7 | — | — |
| 2.50 | 38 | — | — |
| 3.00 | 63 | — | — |
| 3.25 | 87 | — | — |
| 4.25 | 150 | — | — |
| 4.50 | 160 | — | — |
| 5.50 | 169 | Started to be | — |
| | | generated | |
| 6.00 | 177 | Removed | — |
| 6.50 | 180 | Removed | — |
| 7.50 | 190 | Removed | 140 |
| 8.50 | 198 | Removed | 117 |
| 9.00 | 198 | Vacuum | — |
| | | treatment | |
| 10.00 | 198 | Vacuum | 84 |
| | | treatment | |
| 11.00 | 200 | Vacuum | 62 |
| | | treatment | |
| 12.00 | 200 | Vacuum | 51 |
| | | treatment | |

### Examples 10 to 21, Comparative Example 4

First, an unsaturated polyester resin was synthesized. Specifically, 192 g of R-PET flakes (made by Yono PET Bottle Recycle Co., Ltd.) were charged into a 1 L 4-mouth flask equipped with a stirrer, a reflux condenser, a dropping funnel and a gas introducing pipe-possessing thermometer, and melting was carried out by heating to approximately 270° C. using a mantle heater. After that, 0.5 g of dibutyltin oxide was added, and stirring was carried out to uniformity, and then 85 g of propylene glycol was instilled in over approximately 20 minutes. Bumping caused by the instillation of the glycol was not observed. After the instillation had been completed, depolymerization was carried out for 3 hours at 220 to 230° C., and then the temperature was reduced to 160° C., and 98 g of maleic anhydride was added, and moreover the condenser was changed over to a fractional distillation type, and esterification was carried out for 1 hour under a stream of nitrogen at 205 to 210° C. After that, polycondensation was carried out for 1 hour under a reduced pressure of approximately 1330 to 1995 Pa (10 to 15 Torr). The final acid value was 24.7. Next, the stream of nitrogen was changed over to a stream of air, 0.08 g of hydroquinone was added at 160° C., and then dissolution in 291 g of styrene was carried out at 140° C. As a result, a pale yellowish brown transparent unsaturated polyester resin was obtained.

Various additives for preventing cloudiness were added in the amounts shown in Table 9 in terms of parts by mass per 100 parts by mass of the unsaturated polyester resin (Examples 10 to 21), or no such addition was carried out (Comparative Example 4), thus obtaining samples. 20 g of each sample was then taken and put into a test tube of inside diameter 18 mm, the test tubes were left in a constant temperature bath at 25° C., and changes in the appearance were observed.

The results are shown in Table 9.

TABLE 9

| | Additive for | Changes over time | | | | |
|---|---|---|---|---|---|---|
| | preventing | After 1 | After 3 | After 5 | After 10 | After 30 |
| | cloudiness | day | days | days | days | days |
| Example 10 | 0.3 phr | Trans-parent | Slightly | More | Cloudy | — |
| | Lithium | | cloudy | cloudy | | |
| | naphthenate | | | | | |
| Example 11 | 0.1 phr | Trans-parent | Trans-parent | Very | Cloudy | Cloudy |
| | sodium | | | slightly | with sense | |
| | octylate | | | cloudy | of | |
| | | | | | trans-parency | |
| Example 12 | 0.3 phr | Trans-parent | Trans-parent | Trans-parent | Very | Cloudy |
| | sodium | | | | slightly | with sense |
| | octylate | | | | cloudy | of |
| | | | | | | trans-parency |
| Example 13 | 0.5 phr | Trans-parent | Trans-parent | Trans-parent | Trans-parent | Slightly |
| | sodium | | | | | cloudy |
| | octylate | | | | | |
| Example 14 | 0.1 phr | Trans-parent | Trans-parent | Trans-parent | Trans-parent | Slightly |
| | potassium | | | | | cloudy |
| | octylate | | | | | |
| Example 15 | 0.3 phr | Trans-parent | Trans-parent | Trans-parent | Trans-parent | Trans-parent |
| | potassium | | | | | |
| | octylate | | | | | |
| Example 16 | 0.5 phr | Trans-parent | Trans-parent | Trans-parent | Trans-parent | Trans-parent |
| | potassium | | | | | |
| | octylate | | | | | |
| Example 17 | 1.0 phr | Trans-parent | Trans-parent | Trans-parent | Trans-parent | Trans-parent |
| | potassium | | | | | |
| | octylate | | | | | |
| Example 18 | 0.5 phr | Slightly | Cloudy | — | — | — |
| | calcium | cloudy | | | | |
| | octylate | | | | | |
| Example 19 | 0.5 phr | Very | Cloudy | Cloudy | — | — |
| | strontium | slightly | with sense | | | |
| | octylate | cloudy | of | | | |
| | | | trans-parency | | | |
| Example 20 | 0.5 phr | Very | Cloudy | Cloudy | — | — |
| | barium | slightly | with sense | | | |
| | octylate | cloudy | of | | | |
| | | | trans-parency | | | |
| Example 21 | 0.5 phr | Slightly | Cloudy | — | — | — |
| | magnesium | cloudy | | | | |
| | octylate | | | | | |
| Comparative | Blank | Slightly | Cloudy | — | — | — |
| Example 4 | | cloudy | | | | |

As is clear from Table 9, with Examples 10 to 21 of the present invention, an alkali metal organic acid salt was added to the unsaturated polyester resin, and hence the required cloudiness prevention effect was exhibited. In contrast with this, with Comparative Example 4, an alkali metal organic acid salt was not added, and hence undesirable cloudiness occurred.

Note that a system in which 1 part by mass of methyl ethyl ketone peroxide and 0.5 parts by mass of cobalt naphthenate are mixed uniformly into 100 parts by mass of the above unsaturated polyester resin gelated in 37 minutes at 19 to 20° C. The gelation time in the case of adding 0.3 parts by mass of potassium octylate to a resin of the same composition was 29 minutes.

1. A method of depolymerizing polyethylene terephthalate, comprising heating, melting and depolymerizing polyethylene terephthalate to be recycled, wherein the heating, melting and depolymerization reaction of the polyethylene terephthalate to be recycled are carried out all at once using one or a plurality of extruders or using an extruder and a reactor provided at an outlet of the extruder.

2. The method of depolymerizing polyethylene terephthalate according to claim 1, wherein a glycol of a type used as a component of a final product, and a tin-based catalyst such as dibutyltin oxide or a titanium-based catalyst such as tetraisopropoxytitanate that facilitates the depolymerization, are added into the extruder simultaneously or separately during the depolymerization process, and an oligomer having an average molecular weight of not more than 3000 made from the polyethylene terephthalate is produced at a final extruder outlet or a reactor outlet.

3. The method of depolymerizing polyethylene terephthalate according to claim 2, wherein taking the repeat unit of the polyethylene terephthalate to be 1 mol, the amount added of the glycol is made to be 0.1 to 10 mol per 1 mol of the condensed unit of the polyethylene terephthalate, and the amount added of the catalyst is made to be 0.01 to 3 parts by mass per 100 parts by mass of the polyethylene terephthalate used.

4. A method of manufacturing an unsaturated polyester resin, comprising taking a polyethylene terephthalate oligomer obtained using the method according to any one of claims 1 through 3 as a raw material, optionally adding a required amount of a glycol to the oligomer and further advancing decomposition, and then adding an α,β-unsaturated polybasic acid or acid anhydride thereof, further also using another saturated or unsaturated polybasic acid or acid anhydride thereof as necessary, and carrying out polycondensation.

5. A method of depolymerizing a polyester resin, comprising depolymerizing waste PET using a glycol or the like to obtain a depolymerization product, wherein the molecular weight of the depolymerization product is reduced by irradiating the depolymerization product with microwaves.

6. A method of manufacturing a polyester resin, comprising synthesizing a polyester resin by carrying out an esterification reaction using a saturated dibasic acid, an unsaturated polybasic acid and a glycol, wherein the reactants are irradiated with microwaves to raise the temperature and promote the esterification reaction.

7. A method of manufacturing a polyester resin, comprising manufacturing a polyester resin from waste PET by depolymerizing the waste PET using a glycol or the like, and adding an unsaturated polybasic acid such as maleic anhydride to the depolymerization product and heating to carry out an esterification reaction, wherein the temperature of the depolymerization product and the unsaturated polybasic acid is raised by irradiating with microwaves.

8. A method of manufacturing a polyester resin, comprising using a glycol on waste PET to depolymerize the waste PET, and charging an unsaturated polybasic acid such as maleic anhydride into the depolymerization product and heating to carry out an esterification reaction, wherein the esterification reaction is promoted by irradiating the reactants with microwaves.

9. A method of manufacturing a polyester resin, comprising adding an alkali metal organic acid salt to an unsaturated polyester resin containing terephthalic acid.

10. The method of manufacturing a polyester resin according to claim 9, wherein the alkali metal organic acid salt is a sodium organic acid salt or a potassium organic acid salt.

11. The method of manufacturing a polyester resin according to claim 9 or 10, wherein the unsaturated polyester resin containing terephthalic acid is a resin synthesized using a recycled polyethylene terephthalate resin as a raw material.
